(19) United States
(12) Patent Application Publication
(10) Pub. No.: US 2008/0097305 A1
(43) Pub. Date: Apr. 24, 2008

(54) Safety hypodermic syringe

(76) Inventor: Jin-Chou Tsai, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/511,270

(22) Filed: Aug. 29, 2006

(51) Int. Cl. A61M 5/00; A61M 25/00

(52) U.S. Cl. 604/110

(57) ABSTRACT

A safety hypodermic syringe formed of a barrel, a plunger, a retainer, a rubber stopper, a needle holder and a needle is disclosed. The retainer is forced by the plunger into engagement with the needle holder when the plunger is pushed forwards to squeeze a fluid medicine out of the barrel so that the needle holder and the needle are moved carried by the retainer into the inside of the barrel to have spring hooks of the retainer hooked on the rear end edge of the barrel upon a back stroke of the plunger and the needle holder with the needle are kept in the rear side inside the barrel as the plunger is pushed forwards again.

### BACKGROUND OF THE INVENTION

1. Field of the Invention

The present invention relates to a hypodermic syringe and more particularly, to a safety hypodermic syringe, which enables the needle holder and the needle to be received inside the barrel to prevent contamination without breaking the plunger after the service of the safety hypodermic syringe.

2. Description of the Related Art

In recent years, the spreading of AIDS and many other diseases threatens people a lot. These diseases may spread via body fluid or transfusion of blood. Therefore, medical care persons must handle medical implements such as syringe or the like carefully to prevent contamination. In order to prevent contamination, safety hypodermic syringes are developed. A safety hypodermic syringe is known comprising a needle assembly, a barrel, and a plunger with a stopper. The needle assembly is pulled backwards with the plunger after the injection, and then the plunger is broken, keeping the needle assembly inside the barrel. This design of safety hypodermic syringe is still not satisfactory in function because the user must break the plunger after the use of the syringe. Further, the broken part of the plunger greatly increases dimensions of the waste syringe.

### SUMMARY OF THE INVENTION

The present invention has been accomplished under the circumstances in view. It is the main object of the present invention to provide a safety hypodermic syringe, which keeps the needle holder and the needle received inside the barrel to prevent contamination and a re-use of the syringe after the service of the syringe. It is another object of the present invention to provide a safety hypodermic syringe, which keeps the plunger and the needle holder with the needle received inside the barrel after the service of the syringe, reducing the size of the waste syringe.

To achieve these and other objects of the present invention, the safety hypodermic syringe comprises a barrel, the barrel having an inside annular groove around an inside wall of a front neck thereof; a plunger, the plunger having a tubular shank axially slidably inserted into the barrel, an inside annular groove extending around an inside wall of the tubular shank near a front end thereof, and a plurality of longitudinal crevices symmetrically formed on the tubular shank at two sides; a retainer, having a front retaining tip, and a plurality of springy hooks extended from the periphery thereof and respectively radially inserted through the longitudinal crevices of the plunger to the outside of the plunger inside the barrel; a rubber stopper fixedly mounted on the periphery of the retainer and disposed in contact with an inside wall of the barrel; a rubber O-ring mounted on the periphery of the retainer and kept in close contact with the inside wall of the tubular shank of the plunger; and a needle holder holding a needle, the needle holder having a rear retaining hole for receiving the front retaining tip of the retainer, and an outside annular flange engaged into the inside annular groove of the barrel. When the plunger is pushed forward to squeeze a fluid medicine out of the barrel through the needle holder and the needle, the front retaining tip of the retainer is forced into engagement with the rear retaining hole of the needle holder so that the needle holder and the needle are pulled backwards into the inside of the barrel for enabling the springy hooks of the retainer to hook on a rear end edge of the barrel when the plunger is pulled backwards after the service of the safety hypodermic syringe. When the plunger is pushed forwards again after hooking of the springy hooks of the retainer on the rear end edge of the barrel, the needle holder and the needle are kept secured to the inside of the barrel.

### BRIEF DESCRIPTION OF THE DRAWINGS

FIG. 1 is an exploded view of a safety hypodermic syringe in accordance with a first embodiment of the present invention.

FIG. 2 is a sectional assembly view of the safety hypodermic syringe according to the first embodiment of the present invention.

FIG. 3 is a schematic sectional view of the first embodiment of the present invention, showing the needle holder and the needle pulled backwards with the plunger to the inside of the barrel.

FIG. 4 corresponds to FIG. 3, showing the plunger pushed forwards to the front side of the barrel, the needle holder and the needle kept in the rear side inside the barrel.

FIG. 5 is an exploded view of a safety hypodermic syringe in accordance with a second embodiment of the present invention.

FIG. 6 is an exploded view of a safety hypodermic syringe in accordance with a third embodiment of the present invention.

FIG. 7A is an exploded view of a safety hypodermic syringe in accordance with a fourth embodiment of the present invention.

FIG. 7B is a sectional view in an enlarged scale of a part of the plunger shown in FIG. 7A.

FIG. 8 is an exploded view of a safety hypodermic syringe in accordance with a fifth embodiment of the present invention.

FIG. 9 is sectional assembly view of the safety hypodermic syringe according to the fifth embodiment of the present invention.

FIG. 10 is a schematic sectional view of the fifth embodiment of the present invention, showing the needle holder and the needle pulled backwards with the plunger to the inside of the barrel.

FIG. 11 corresponds to FIG. 10, showing the plunger pushed forwards to the front side of the barrel, the needle holder and the needle kept in the rear side inside the barrel.

FIG. 12 is an exploded view of a safety hypodermic syringe in accordance with a sixth embodiment of the present invention.

FIG. 13 is sectional assembly view of the safety hypodermic syringe according to the sixth embodiment of the present invention.

FIG. 14 is a schematic sectional view of the sixth embodiment of the present invention, showing the plunger pushed forwards to the front side of the barrel, the needle holder and the needle kept in the rear side inside the barrel.

FIG. 15 is an exploded view of a safety hypodermic syringe in accordance with a seventh embodiment of the present invention.

### DETAILED DESCRIPTION OF THE PREFERRED EMBODIMENT

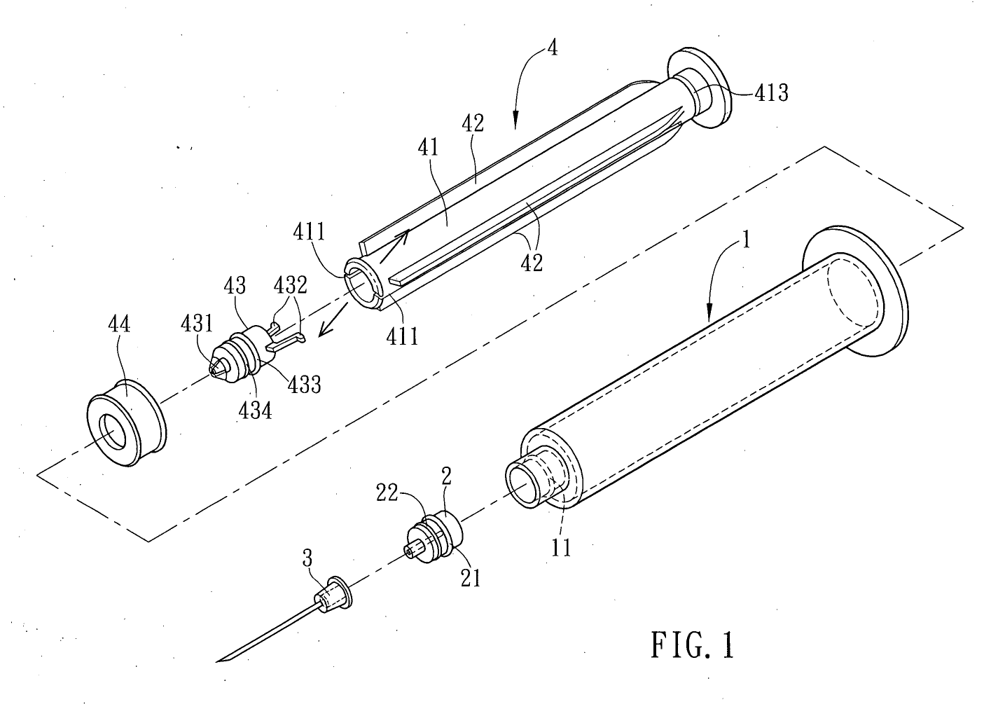

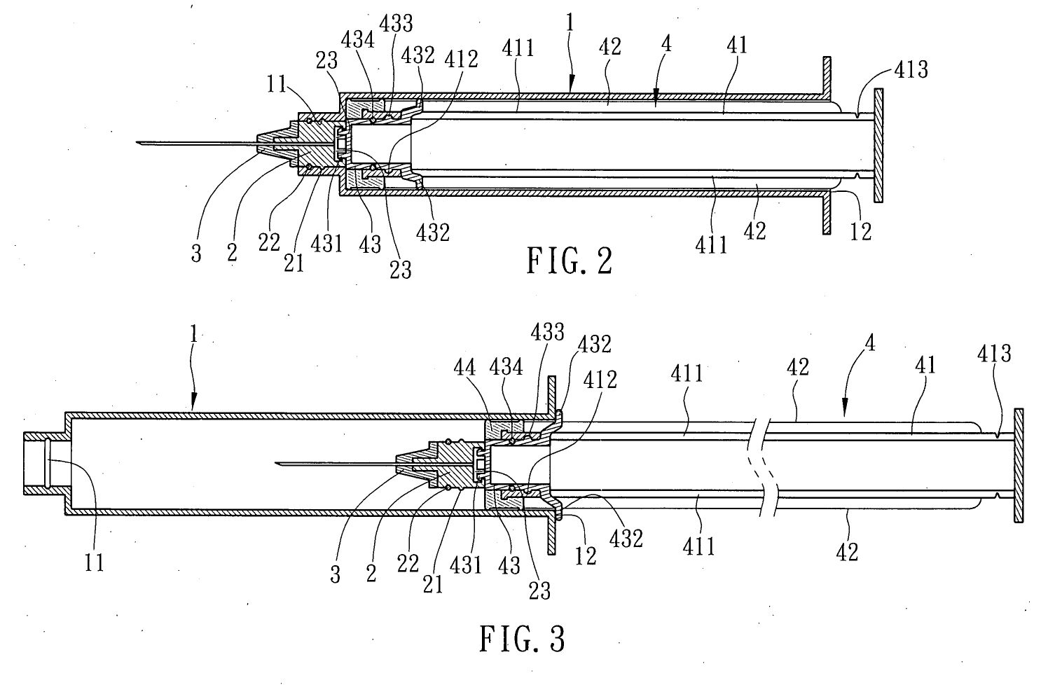

Referring to FIGS. 1 and 2, a safety hypodermic syringe in accordance with the present invention is shown comprised of a barrel 1, a plunger 4, a retainer 43, a rubber stopper 44, a needle holder 2, and a needle 3.

The barrel 1 has an inside annular groove 11 around the inside wall of the front neck thereof.

The plunger 4 has a tubular shank 41 axially slidably inserted from the rear side of the barrel 1 into the inside of the barrel 1, an inside annular groove 412 extending around the inside wall of the tubular shank 41 near the front end, two longitudinal crevices 411 symmetrically formed on the peripheral wall of the tubular shank 41 at two sides, and two longitudinal ribs 42 equiangularly spaced around the periphery of the tubular shank 41 and disposed in contact with the inside wall of the barrel 1.

The retainer 43 is inserted through the rubber stopper 44, having a front retaining tip 431 for securing the needle holder 2, an outside annular flange 433 engaged into the inside annular groove 412 of the plunger 4, and two springy hooks 432 extended from the periphery thereof at two sides and respectively radially inserted through the longitudinal crevices 411 of the plunger 4 to the outside of the plunger 4 inside the barrel 1.

Further, a rubber O-ring 434 is mounted on the periphery of the retainer 43 and kept in close contact with the inside wall of the tubular shank 41 of the plunger 4.

The needle holder 2 has a rear retaining hole 23 for receiving the front retaining tip 431 of the retainer 43, and an outside annular flange 21 engaged into the inside annular groove 11 of the barrel 1. Further, a rubber O-ring 22 is mounted on the periphery of the needle holder 2 and kept in close contact with the inside wall of the barrel 1. The needle 3 is fixedly fastened to the front side of the needle holder 2 and extending to the outside of the barrel 1.

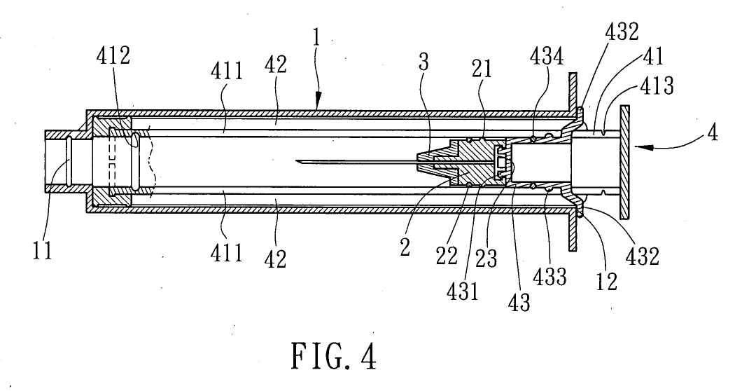

When the operator pushed the plunger 4 to the front side of the barrel 1 to squeeze the fluid medicine out of the barrel 1 into the patient's body, the front retaining tip 431 of the retainer 43 is forced into engagement with the rear retaining hole 23 of the needle holder 2. After the service of the safety hypodermic syringe, the operator pulls the plunger 4 backwards. At this time, the needle holder 2 and the needle 3 are carried with the plunger 4 backwards to the inside of the barrel 1. After the plunger 4 has been pulled backwards to the extent as shown in FIG. 3, the springy hooks 432 are hooked on the rear end edge 12 of the barrel 1. At this time, the operator can push the plunger 4 forwards again, keeping the needle holder 2 and the needle 3 secured to the rear side inside the barrel 1 (see FIG. 4).

Further, the plunger 4 has a breaking neck 413 near the rear side. After the needle holder 2 and the needle 3 have been secured to the rear side inside the barrel 1, the operator can break the breaking neck 413.

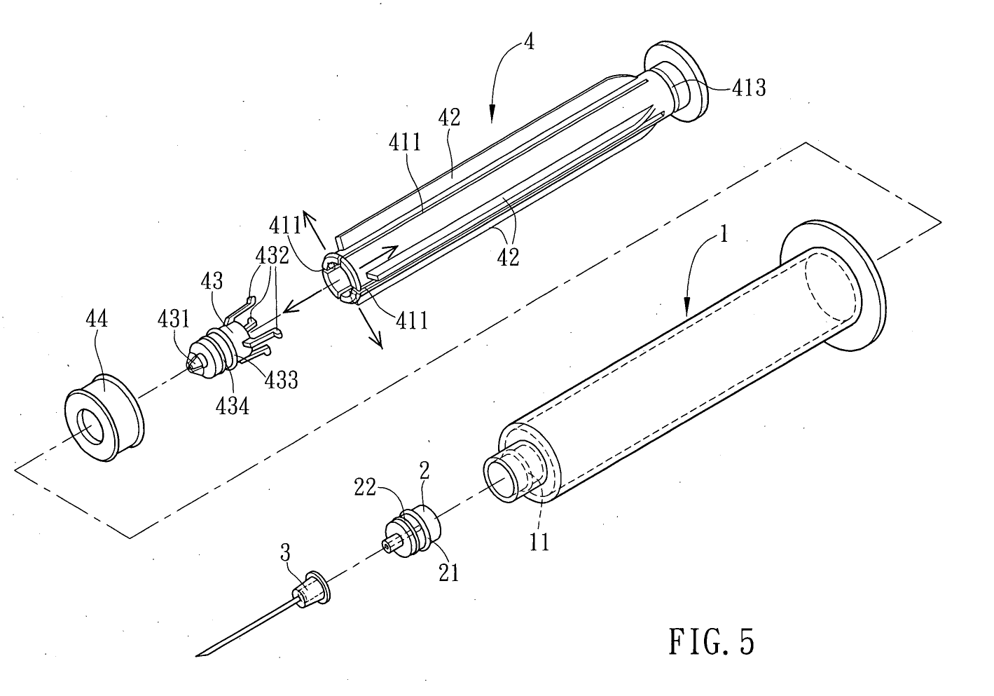

FIG. 5 is an exploded view of a safety hypodermic syringe in accordance with a second embodiment of the present invention. This embodiment is substantially similar to the aforesaid first embodiment with the exception of the number of the springy hooks 432 of the retainer 43 and the number of the longitudinal crevices 411 of the plunger 4.

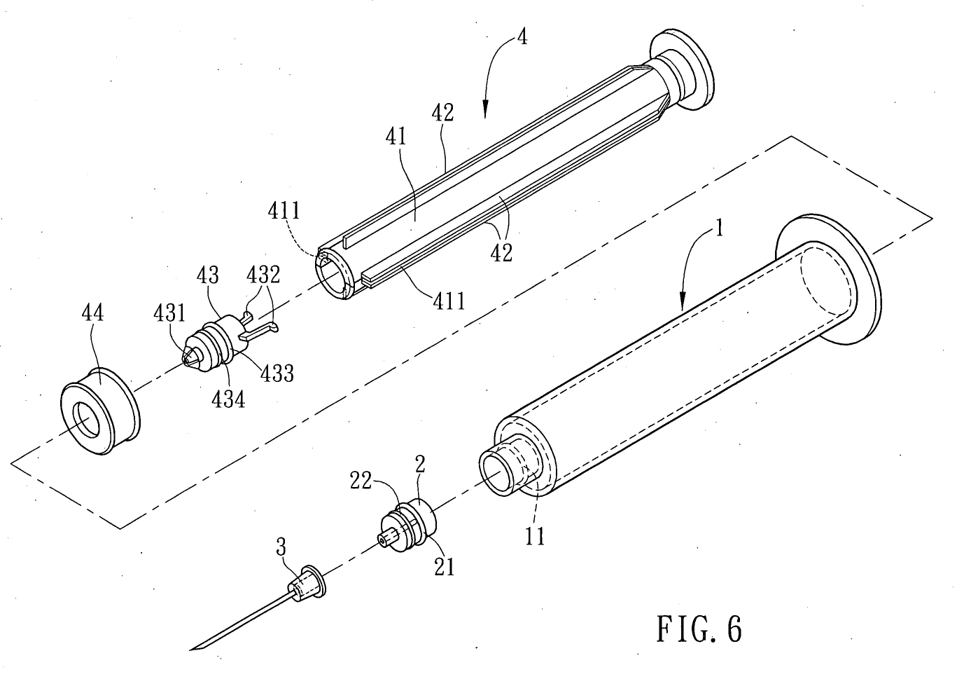

FIG. 6 is an exploded view of a safety hypodermic syringe in accordance with a third embodiment of the present invention. This embodiment is substantially similar to the aforesaid first embodiment with the exception that the plunger 4 has pairs of longitudinal ribs 42 equiangularly spaced around the periphery. The longitudinal ribs 42 stabilize axial movement of the plunger 4 relative to the barrel, prohibiting the springy hooks 432 of the retainer 43 from touching the inside wall of the barrel 1 before reaching the rear side of the barrel 1.

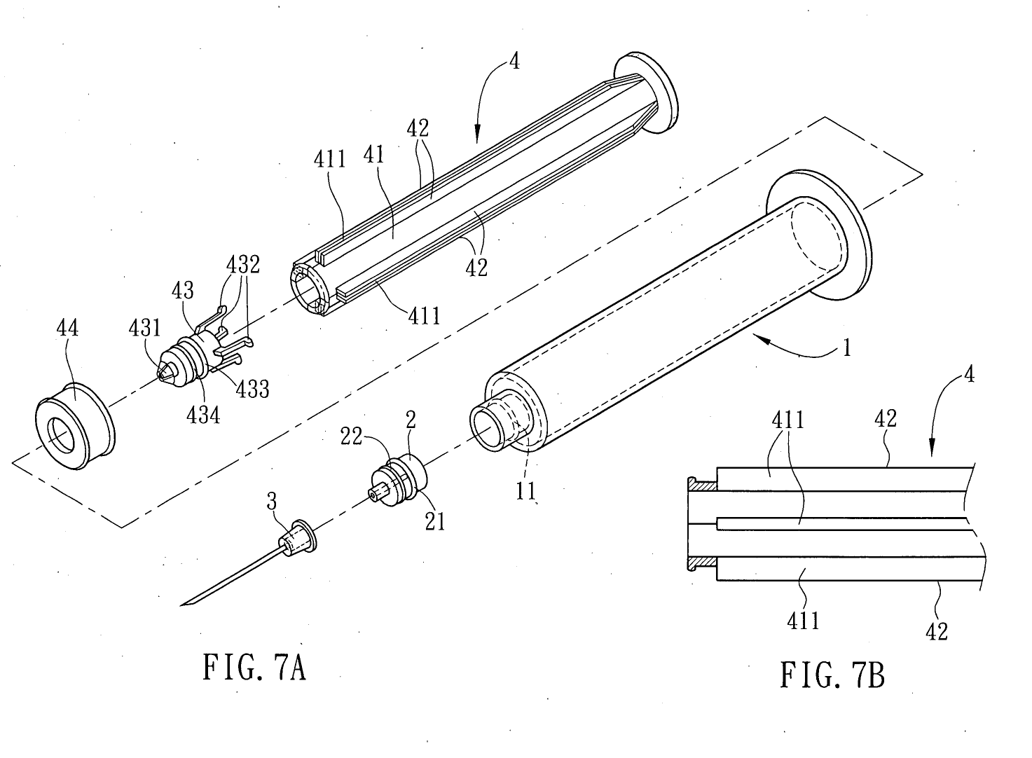

FIG. 7A is an exploded view of a safety hypodermic syringe in accordance with a fourth embodiment of the present invention. This embodiment is substantially similar to the aforesaid third embodiment with the exception of the number of the springy hooks 432 of the retainer 43 and the number of the longitudinal crevices 411 of the plunger 4. FIG. 7B is a sectional view in an enlarged scale of a part of the plunger shown in FIG. 7A.

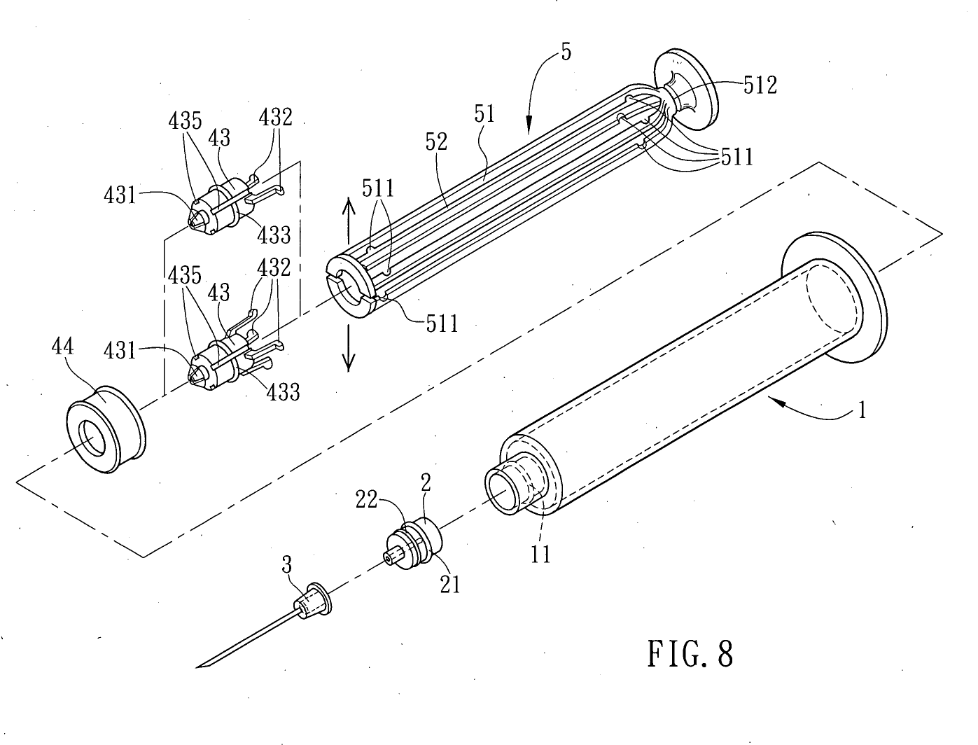

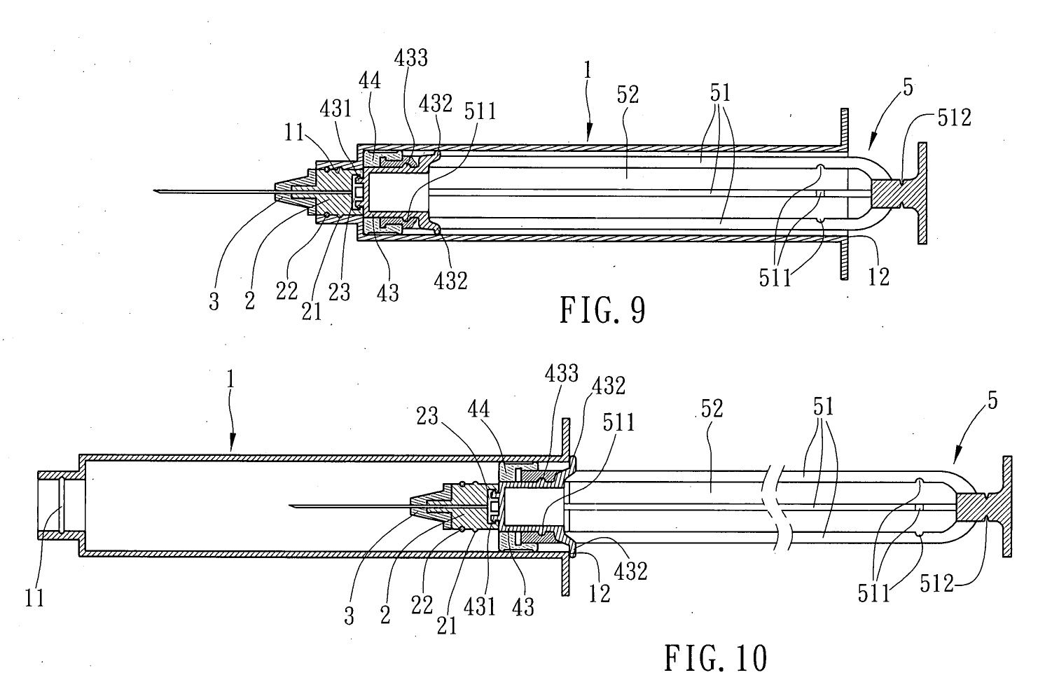

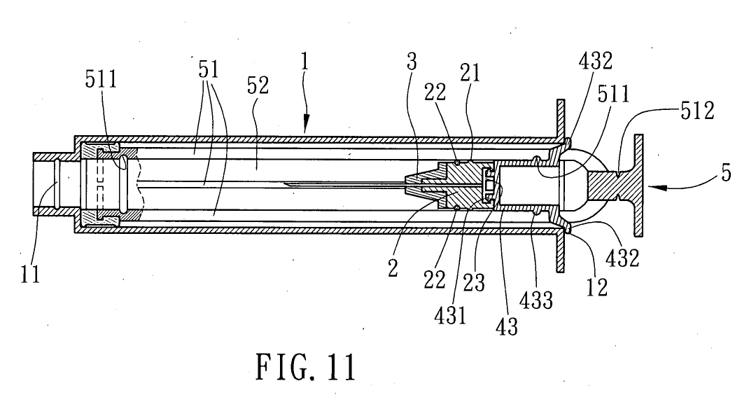

FIGS. 8˜11 show a safety hypodermic syringe in accordance with a fifth embodiment of the present invention. According to this embodiment, the plunger 5 has a breaking neck 512 near the rear side, a plurality of longitudinal crevices 52 cut through the periphery, a plurality of longitudinal ribs 51 equiangularly spaced around the inside wall, and two inside annular grooves 511 extending around the inside wall near the front and rear sides.

The retainer 43 has a plurality of longitudinal grooves 435, which receive the longitudinal ribs 51 of the plunger 5 to guide movement of the retainer 43 relative to the barrel 1, an outside annular flange 433 extending around the periphery for selectively engaging the inside annular grooves 511 to secure the retainer 43 to the front or rear side inside the barrel 1, a plurality of springy hooks 432 radially extending out of the longitudinal crevices 52 of the plunger 5, and a front retaining tip 431 for securing the retainer 2. This embodiment works in the same way as the aforesaid various different embodiments of the present invention.

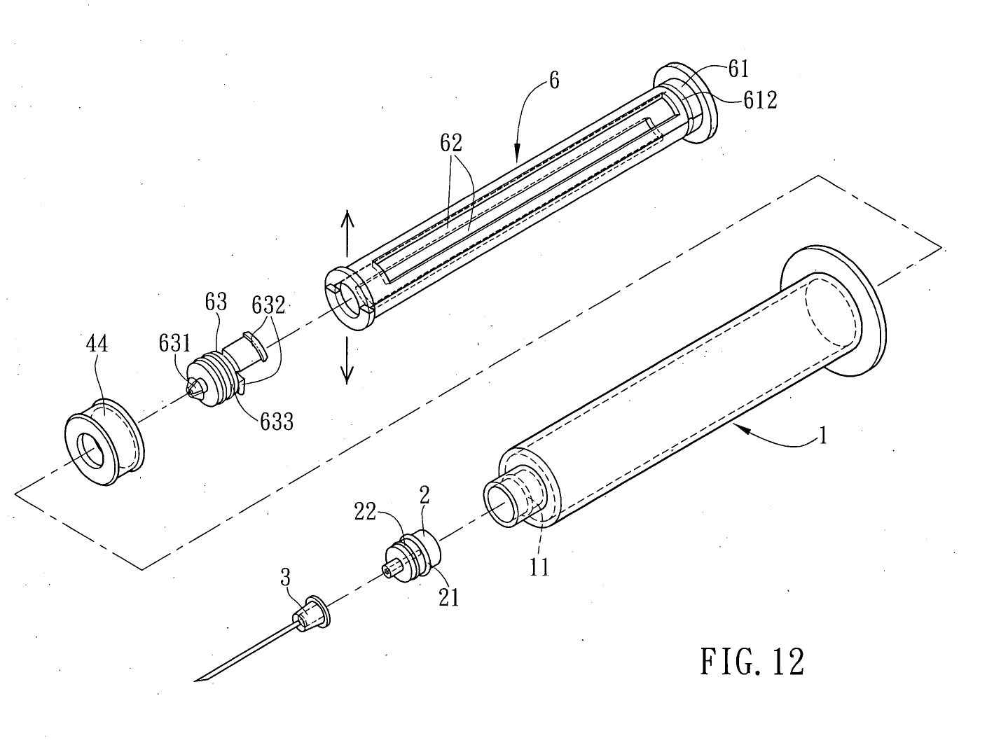

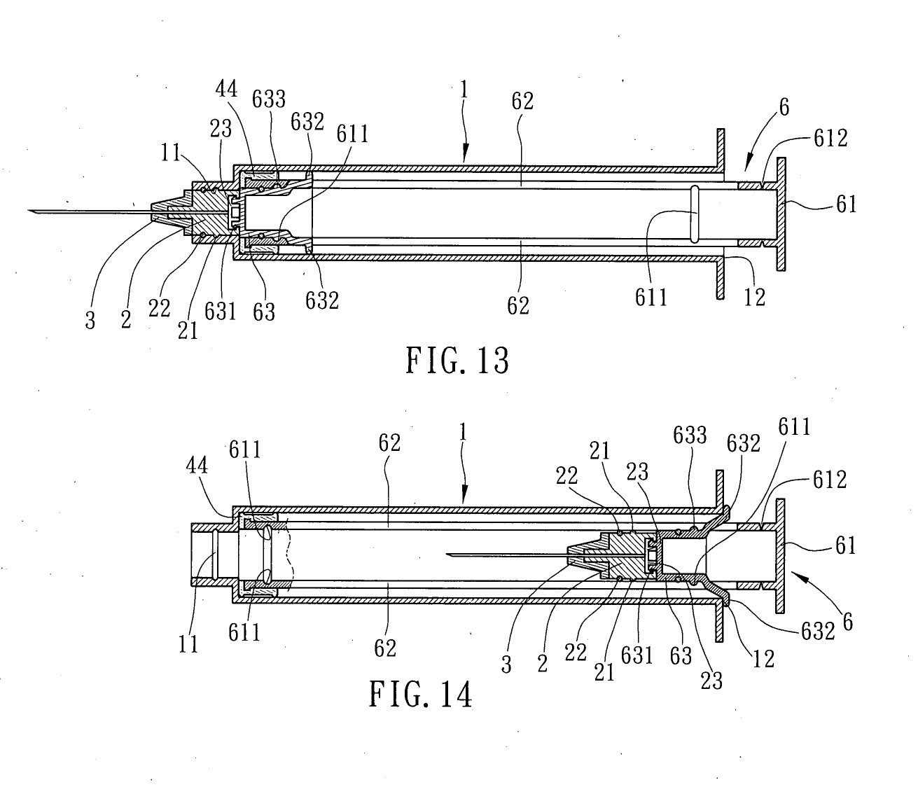

FIGS. 12˜14 show a safety hypodermic syringe in accordance with a sixth embodiment of the present invention.

According to this embodiment, the tubular shank 61 of the plunger 6 has a breaking neck 612 near the rear side, two longitudinal crevices 62 cut through the periphery, and two inside annular grooves 611 respectively extending around the inside wall near the front and rear sides. The retainer 63 has a front retaining tip 631 for securing the retainer 2, an outside annular flange 633 for selectively engaging the inside annular grooves 611 to secure the retainer 63 to the front or rear side inside the barrel 1, and a plurality of springy hooks 632 radially extending out of the longitudinal crevices 62 of the plunger 6. This embodiment is substantially similar to the aforesaid first embodiment with the exception of the shape of the springy hooks 632 and the shape of the longitudinal crevices 62. Therefore, this embodiment works in the same way as the aforesaid various different embodiments of the present invention.

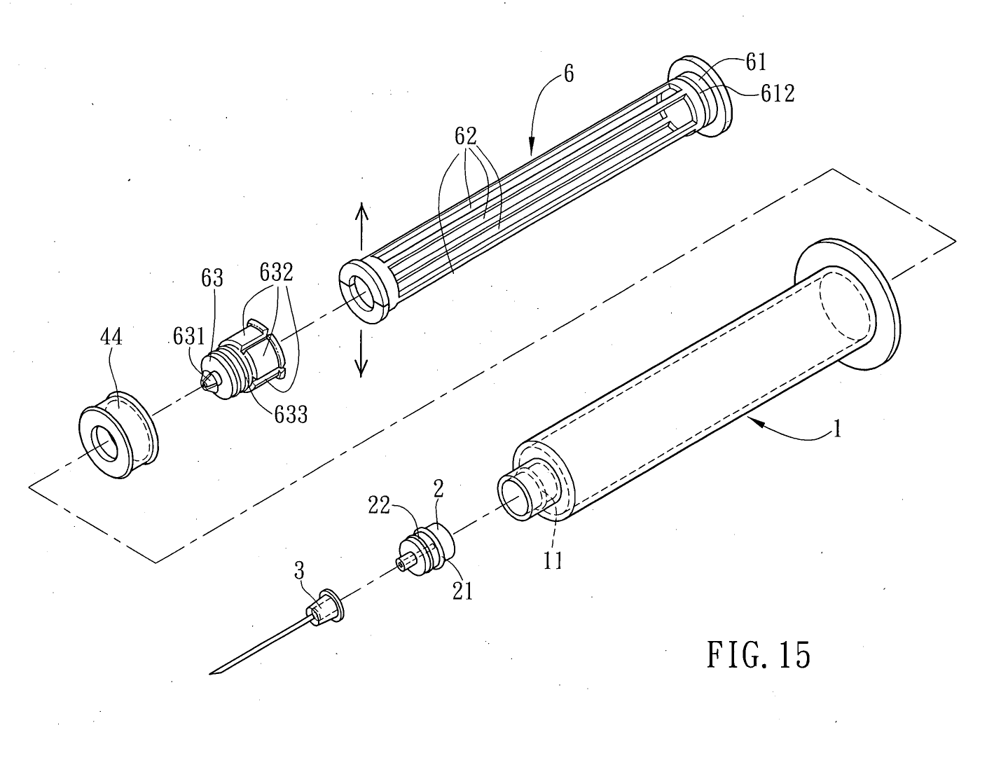

FIG. 15 is an exploded view of a safety hypodermic syringe in accordance with a seventh embodiment of the present invention. This embodiment is substantially similar to the aforesaid sixth embodiment of the present invention with the exception of the number of the springy hooks 632 and the number of the longitudinal crevices 62.

A prototype of safety hypodermic syringe has been constructed with the features of FIGS. 1˜15. The safety hypodermic syringe functions smoothly to provide all the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A safety hypodermic syringe comprising:
a barrel, said barrel having an inside annular groove around an inside wall of a front neck thereof;
a plunger, said plunger having a tubular shank axially slidably inserted into said barrel, an inside annular groove extending around an inside wall of said tubular shank near a front end thereof, and a plurality of longitudinal crevices symmetrically formed on said tubular shank at two sides;
a retainer, having a front retaining tip, and a plurality of springy hooks extended from the periphery thereof and respectively radially inserted through the longitudinal crevices of said plunger to the outside of said plunger inside said barrel;
a rubber stopper fixedly mounted on the periphery of said retainer and disposed in contact with an inside wall of said barrel;
a rubber O-ring mounted on the periphery of said retainer and kept in close contact with the inside wall of said tubular shank of said plunger; and
a needle holder holding a needle, said needle holder having a rear retaining hole for receiving the front retaining tip of said retainer, and an outside annular flange engaged into the inside annular groove of said barrel;
wherein when said plunger is pushed forward to squeeze a fluid medicine out of said barrel through said needle holder and said needle, said front retaining tip of said retainer is forced into engagement with the rear retaining hole of said needle holder so that said needle holder and said needle are pulled backwards into the inside of said barrel for enabling the springy hooks of said retainer to hook on a rear end edge of said barrel when said plunger is pulled backwards after the service of the safety hypodermic syringe; when said plunger is pushed forwards again after hooking of the springy hooks of said retainer on the rear end edge of said barrel, said needle holder and said needle are kept secured to the inside of said barrel.

2. The safety hypodermic syringe as claimed in claim 1, wherein said plunger has a first inside annular groove and a second inside annular groove respectively disposed near front and rear sides of said tubular shank; said retainer has an outside annular flange selectively engageable into the first inside annular groove and second inside annular groove of said plunger.

3. The safety hypodermic syringe as claimed in claim 1, wherein said plunger has a plurality of longitudinal ribs equiangularly spaced around the periphery thereof and disposed in contact with an inside wall of said barrel.

4. The safety hypodermic syringe as claimed in claim 1, wherein said shank of said plunger has a breaking neck near a rear end thereof.

5. A safety hypodermic syringe comprising:
a barrel, said barrel having an inside annular groove around an inside wall of a front neck thereof;
a plunger, said plunger having a tubular shank axially slidably inserted into said barrel, and a plurality of longitudinal ribs equiangularly spaced around the periphery of said tubular shank and disposed in contact with an inside wall of said barrel, a plurality of longitudinal slots cut through the periphery of said tubular shank and respectively separated by said longitudinal ribs;
a retainer, having a front retaining tip, a rear retaining hole, and a plurality of springy hooks extended from the periphery thereof and respectively radially inserted through the longitudinal open spaces of said plunger to the outside of said plunger inside said barrel;
a rubber stopper fixedly mounted on the periphery of said retainer and disposed in contact with an inside wall of said barrel;
a rubber O-ring mounted on the periphery of said retainer and kept in close contact with the inside wall of said tubular shank of said plunger; and
a needle holder holding a needle, said needle holder having a rear retaining hole for receiving the front retaining tip of said retainer, and an outside annular flange engaged into the inside annular groove of said barrel;
wherein when said plunger is pushed forward to squeeze a fluid medicine out of said barrel through said needle holder and said needle, said front retaining tip of said retainer is forced into engagement with the rear retaining hole of said needle bolder so that said needle holder and said needle are pulled backwards into the inside of said barrel for enabling the springy hooks of said retainer to hook on a rear end edge of said barrel when said plunger is pulled backwards after the service of the safety hypodermic syringe; when said plunger is pushed forwards again after hooking of the springy hooks of said retainer on the rear end edge of said barrel, said needle holder and said needle are kept secured to the inside of said barrel.

6. The safety hypodermic syringe as claimed in claim 5, wherein said plunger has a first inside annular groove and a second inside annular groove respectively disposed near front and rear sides of said tubular shank; said retainer has an outside annular flange selectively engageable into the first inside annular groove and second inside annular groove of said plunger.

7. The safety hypodermic syringe as claimed in claim 5, wherein said plunger has a plurality of longitudinal grooves equiangularly spaced around an inside wall thereof and disposed in contact with an inside wall of said barrel; said retainer has a plurality of longitudinal ribs equiangularly spaced around the periphery and respectively engaged into the longitudinal grooves of said plunger.

8. The safety hypodermic syringe as claimed in claim 5, wherein said shank of said plunger has a breaking neck near a rear end thereof.

9. The safety hypodermic syringe as claimed in claim 5, wherein said retainer has an outer diameter smaller than the inner diameter of the tubular shank of said plunger.
